(19) United States
(12) Patent Application Publication
(10) Pub. No.: US 2025/0185970 A1
(43) Pub. Date: Jun. 12, 2025

(54) ELECTRODE STRUCTURE AND ELECTRODE MODULE FOR NEURAL PROBE

(71) Applicants: NFORMARE INC., Seoul (KR); YONSEI UNIVERSITY UNIVERSITY-INDUSTRY FOUNDATION, Seoul (KR)

(72) Inventors: Heon-Jin CHOI, Seoul (KR); Jae-Suk SUNG, Seoul (KR); Young-Cheol CHAE, Seoul (KR)

(21) Appl. No.: 18/840,967

(22) PCT Filed: Jan. 27, 2023
(86) PCT No.: PCT/KR2023/001267

§ 371 Date: Aug. 23, 2024

(30) Foreign Application Priority Data
Feb. 25, 2022 (KR) 10-2022-0024968

(51) Int. Cl. A61B 5/294; A61B 5/388

(52) U.S. Cl. CPC A61B 5/294; A61B 5/388

(57) ABSTRACT

The present invention provides the electrode structure of a neural probe including electrodes for bio-signal measurement and stimulation. As an embodiment, the present invention provides an electrode structure for a neural probe that measures bio-logical signals or applies stimulation, the electrode structure including: a substrate; electrodes formed on at least one surface of the substrate; and a wiring formed on the substrate and connected to the electrodes; wherein the electrodes comprise one or more measurement electrodes connected to a measurement circuit and one or more stimulation electrodes connected to a stimulation circuit.

### TECHNICAL FIELD

The present invention relates to an electrode structure and electrode module for a neural probe including a measurement electrode and a stimulation electrode.

### BACKGROUND ART

A neural probe refers to a micro-electrode element for neural interface that can measure neural signals at the final stage of an electronic medicine product or stimulate nerves by allowing current to flow thereto. A neural probe includes a micro-electrode element connected to a circuit module or a circuit module operating in conjunction with a micro-electrode element. Using a neural probe has the effect of treating nerve-related problems in the short or long term.

For example, in the past, in order to measure brain signals, metal electrodes are inserted into the corresponding parts of the brain and measure action potentials. However, the brains of vertebrates are so complex that they consist of about 100 billion neurons, so that in order to elucidate brain circuits, there is required a system for measuring neural signals from multiple parts of the brain at the same time. For this purpose, neural probes are used to measure brain signals.

An electrode array for measuring signals is integrated at the end of the body of a neural probe, the signals measured from electrodes are transmitted to the outside through a wire formed along the probe, and dozens of electrodes can be integrated into a single probe body. Accordingly, signals can be measured simultaneously from multiple nerves. The form of the signals output in this case may be electrical signals, optical signals, or the like, and there is no particular limitation on the form of the signals. In addition, the probe can be equipped with a drug injection channel, and thus, can serve as a medium for delivering a drug.

The body of a neural probe is mainly made using a silicon material. This is adopted in an MEMS-type probe 1 shown in FIG. 1. The probe 1 includes an electrode part 10 configured to be inserted into a nerve or cell, and a connection part 20 connected to the electrode part 10 and provided with terminals for connecting a main body and the electrode part 10. The body formed on a substrate made of a silicon material has a bulk shape having a specific thickness. There is a problem in that the electrode part 10 is easily broken when inserted and maintained in the human body or when handled, and also has a lack of flexibility, which acts as a limiting factor in selecting an effective location in the human body. Therefore, improvements are required for this.

The electrodes of a neural probe serve to measure bio-signals of nerves or cells or transmit stimuli to nerves or cells. The electrode for the former and the electrode for the latter differ from each other in terms of electrode size, impedance characteristics, etc. Accordingly, a neural probe for measuring bio-signals and a neural probe for transmitting stimuli are manufactured and used separately. The reason for this is that it is technically difficult to incorporate two types of electrodes having different electrode sizes and different impedance characteristics into a single neural probe.

In particular, when neural probes are operated separately, it is difficult to specify a location when a bio-signal measurement location and a stimulation delivery location are the same, and there are limitations in measuring a bio-signal or effectively delivering stimulation. In other words, there is a problem in that it is difficult to appropriately respond to the need to measure a bio-signal and provide immediate stimulation when an abnormality occurs to provide real-time treatment.

### DISCLOSURE

### Technical Problem

The present invention is intended to enable precise stimulation treatment by forming and operating different bio-signal measurement and stimulation electrodes simultaneously in a single neural probe, particularly in an electrode probe region to be inserted into the human body, so that a measurement location and a stimulation location are considerably close to each other or exactly match each other.

### Technical Solution

In order to overcome the above-described technical problem, the present invention provides the following electrode structures and electrode modules for a neural probe.

In an embodiment, the present invention provides an electrode structure for a neural probe that measures bio-logical signals or applies stimulation, the electrode structure including: a substrate; electrodes formed on at least one surface of the substrate; and a wiring formed on the substrate and connected to the electrodes; wherein the electrodes comprise one or more measurement electrodes connected to a measurement circuit and one or more stimulation electrodes connected to a stimulation circuit.

In an embodiment, the measurement electrodes may be arranged on one surface of the substrate, the stimulation electrodes may be arranged on an opposite surface of the substrate, and at least some of the measurement electrodes and at least some of the stimulation electrodes may be arranged at corresponding locations with the substrate interposed therebetween.

In an embodiment, the present invention provides an electrode structure for a neural probe that measures bio-logical signals or applies stimulation, the electrode structure including: a substrate; electrodes formed on at least one surface of the substrate; and a wiring formed on the substrate and connected to the electrodes; wherein the electrodes include one or more measurement and stimulation electrodes connected to both a measurement circuit and a stimulation circuit.

In an embodiment, switching elements configured to selectively allow and block connection between the measurement and stimulation electrodes and the measurement circuit or the stimulation circuit may be arranged between the measurement circuit and the stimulation circuit, and the measurement and stimulation electrodes.

In an embodiment, the electrode structure may further include a heat dissipation layer exposed out of the outer surface of the electrode structure.

In an embodiment, the electrode structure may further include an insulation layer configured to cover parts of the substrate and the electrodes.

In an embodiment, the insulation layer may include through holes exposing the electrodes, and the area of the through holes may be smaller than the area of the corresponding electrodes.

In an embodiment, the electrodes may each have depression-protrusion structures formed on the exposed surfaces thereof.

In an embodiment, the present invention provides an electrode module for a neural probe, the electrode module including: the above-described electrode structure; and a main body including a circuit part connected to the electrodes of the electrode structure; wherein the circuit part and the electrode structure are formed on the same substrate.

In an embodiment, the present invention provides an electrode module for a neural probe, the electrode module including: the above-described electrode structure for a neural probe; and a main body including a circuit part connected to the measurement and stimulation electrodes; wherein the circuit part includes the measurement circuit and the stimulation circuit.

### Advantageous Effects

According to the present invention described above, different bio-signal measurement and stimulation electrodes are formed and operated simultaneously in a single neural probe, particularly in an electrode probe region to be inserted into the human body, so that a measurement location and a stimulation location are considerably close to each other or exactly match each other, with the result that the effect of enabling precise stimulation treatment may be expected.

### DESCRIPTION OF DRAWINGS

FIG. 1 is a drawing showing a conventional MEMS-type neural probe;

FIGS. 2 and 3 are drawings showing the electrode structures of neural probes according to embodiments of the present invention;

FIG. 4 shows a partial sectional view and plan view of the electrode structure of a neural probe according to an embodiment of the present invention;

FIGS. 5 and 6 are drawings showing the simulation results of a case where there is no insulation layer in an electrode and a case where an electrode is covered with an insulation layer including a through hole;

FIG. 7 shows a partial sectional view and plan view of the electrode structure of a neural probe according to another embodiment of the present invention;

FIG. 8 is a drawing showing the simulation results of a case where one electrode is covered with an insulation layer including a plurality of through holes;

FIG. 9 is a schematic diagram of an electrode module including the electrode structure of a neural probe according to an embodiment of the present invention;

FIG. 10 is a schematic diagram of the electrode structure of a neural probe according to another embodiment of the present invention;

FIG. 11 is a schematic diagram of the electrode structure of a neural probe according to another embodiment of the present invention;

FIGS. 12 and 13 are schematic diagrams of an electrode module including the electrode structure of the neural probe according to FIG. 8 of the present invention, wherein FIG. 12 is a schematic diagram of the electrode module viewed from one surface and FIG. 13 is a schematic diagram of the electrode module viewed from the opposite surface;

FIGS. 14 to 17 are schematic diagrams of the electrode modules of neural probes according to embodiments of the present invention;

FIG. 18 is a schematic diagram of the electrode part of a neural probe according to another embodiment of the present invention;

FIGS. 19 and 20 are sectional views of the electrode structures of neural probes according to embodiments of the present invention;

FIG. 21 is a sectional diagram of the electrode structure of a neural probe according to another embodiment of the present invention; and

FIG. 22 is a sectional diagram of the electrode structure of a neural probe according to another embodiment of the present invention.

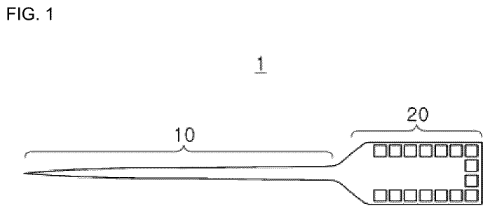

*Description of Reference Symbols*

1: neural probe
10: electrode part
11: base film
15: insulation layer
16: through hole
17: heat dissipation layer
20: connection part
30: electrode
30a: measurement electrode
30b: stimulation electrode
30c: measurement and stimulation electrode
40: wiring
50: main body
60: switching element
70: measurement circuit
80: stimulation circuit
90: measurement and stimulation circuit
100: electrode module

### MODE FOR INVENTION

Referring to the accompanying drawings, preferred embodiments will be described in detail below so that those having ordinary knowledge in the art to which the present invention pertains can easily practice the present invention. However, in the following detailed description of preferred embodiments of the present invention, when it is determined that a specific description of a related known function or configuration may unnecessarily obscure the gist of the present invention, the detailed description will be omitted. Furthermore, the same symbols are used throughout the drawings for the parts that perform similar functions and operations. Moreover, in the present specification, terms such as ‘upper,’ ‘upper part,’ ‘upper surface,’ ‘lower,’ ‘lower part,’ ‘lower surface,’ ‘side surface’, etc. are based on the drawings, and in reality, they may vary depending on the direction in which elements or components are arranged.

In addition, throughout the specification, when a part is described as being ‘connected’ to another part, this includes not only a case where they are ‘directly connected to each other,’ but also a case where they are ‘indirectly connected’ to each other with another element interposed therebetween. Furthermore, ‘including’ a component means that, unless otherwise specifically stated, another component is not excluded, but may be further included.

In a neural probe, a module for measuring bio-signals is usually installed independently and separately from the electrode probe and is operated in the state of being electrically connected to the electrode probe. However, there is a problem in that the size of the neural probe system, which includes a bio-signal measurement module, a stimulation module and an electrode probe, increases and the structure of the system becomes complicated. In particular, when the electrode probe and the bio-signal measurement module are constructed separately, the bio-signal measurement module needs to be worn together with the electrode probe in the state in which the electrode probe is inserted into the human body, and there is a possibility that measurement may be interrupted due to artificial damage to the wiring connecting the electrode structure and the module. In this case, there is a problem in that the effective operation of the neural probe is hindered.

To this end, the present invention provides an electrode structure including a measurement electrode and a stimulation electrode, or provides the electrode structure of a neural probe including a measurement and stimulation electrode for performing measurement or stimulation, in order to enable effective operation while reducing the size of the neural probe.

The present invention provides not only an electrode structure, but also an electrode module that is connected to a circuit part connected to electrodes.

In addition, in the case where such a neural probe is employed, when electrical or optical stimulation is applied to a nerve or cell using the neural probe, heat is generated locally. When the temperature of the heat is equal to or higher than 42° C., the deformation or destruction of a nerve or cell that comes into contact with or is adjacent to the neural probe may begin, so that there is a possibility that it may cause fatal consequences for a patient who wears the neural probe. Accordingly, there is a need for the development of a technology using a method of removing generated heat or minimizing the generation of heat.

There is no specific alternative for removing heat, and in particular, a heat dissipation structure specialized for a neural probe has not been proposed. In addition, in the case of a method of minimizing the generation of heat, the generation of heat may be suppressed by reducing input power. However, efficient stimulation is proportional to the input power, so that the efficiency of stimulation is also reduced.

To this end, the electrode structure of the present invention includes a heat dissipation layer exposed to the outside in an embodiment, and may prevent the deformation of nerves or cells attributable to excessive heating to a specific temperature or higher by dissipating generated heat through the heat dissipation layer.

In addition, the size and exposed area of the electrodes formed in the neural probe are important factors in determining the impedance. As the size of the electrodes increases, the impedance becomes higher. Since the front surfaces of the electrodes are usually exposed, the common direction of the conventional technology has been to form the electrodes so that the size of the electrodes is increased. However, there is a limitation on increasing the size of the electrodes. Furthermore, as the size of the electrodes increase, it becomes more difficult to specify a bio-signal measurement target nerve or cell. Accordingly, there is difficulty in identifying the problem of a specific nerve or cell and treating it appropriately.

In the present invention, through holes are formed in an insulation layer and electrodes are exposed through the through holes in an embodiment, so that accurate measurement and stimulation are enabled, and the electrode structure of a neural probe is improved, so that stimulation and biosignal measurement functions are implemented in a single neural probe. Accordingly, the present invention provides an electrode structure for a neural probe that matches a bio-signal measurement location and a stimulation location as much as possible.

Furthermore, in the present invention, a measurement circuit is directly mounted on one side of the area where the electrode wiring of a neural probe is formed in an embodiment. Accordingly, there is no need to construct a separate external measurement device, which is convenient, and an overall system including the neural probe may be made lighter, simpler, and smaller and may also be advantageous for mass production.

Furthermore, in the present invention, a bio-signal is measured through a measurement electrode and stimulation is immediately applied through a stimulation electrode when an abnormality occurs in an embodiment, so that it may be possible to implement a feedback system that treats a nerve or cell in real time.

Furthermore, in the present invention, the size of electrodes is maximized and parts of the electrodes are covered with an insulation layer to reduce the exposed area of the electrodes in an embodiment, so that the impedance of the electrodes can be minimized and based on this, the accuracy of bio-signal measurement can be increased. Furthermore, the exposed area of the electrodes is small, so that the region of a nerve or cell for the measurement of a bio-signal can be precisely specified, with the result that the measurement or stimulation of the corresponding region can be performed precisely.

Moreover, the exposed areas of electrodes are reduced, so that energy is concentrated on the exposed areas, with the result that the concentrated energy can be used to stimulate a specific nerve or cell to further increase the effect of stimulation.

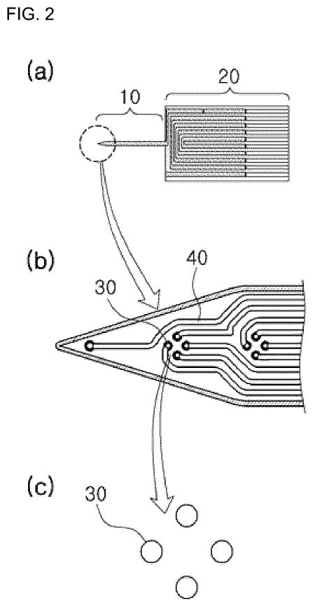

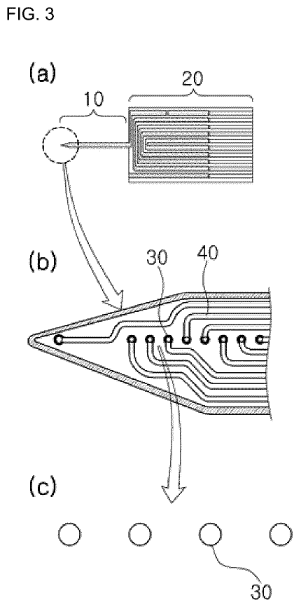

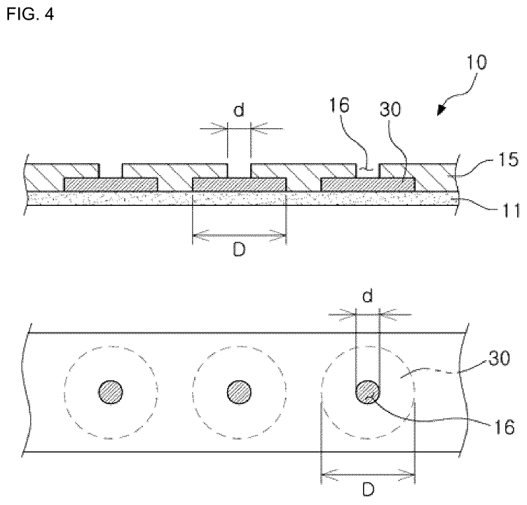

As shown in FIGS. 2 and 3, the electrode structure of a neural probe according to an embodiment of the present invention includes an electrode part 10 and a connection part 20, and the electrode part 10 and the connection part 20 are implemented by using a base film in the form of a film as a substrate. The electrode part 10 includes a base film 11 (see FIG. 4), electrodes 30 formed on the base film 11, and a wiring 40 connected to the electrodes 30 and connected to a measurement circuit 70 (see FIG. 13) or stimulation circuit 80 (see FIG. 13) configured in the outside of the electrode structure, the electrode part 10, or the connection part 20. The electrode part 10 may be inserted into a nerve or the like, and the electrode part 10 may be connected to the connection part 20 for connection to the main body of a separate electrode module or may be directly connected to the main body of the electrode module. In the case of the connection part 20, terminals may be included such that they can be connected to the main body of the electrode module by wire bonding or the like.

The electrodes 30 may be arranged in a tetrode-type arrangement as shown in FIG. 2(c) or a linear-type arrangement as shown in FIG. 3(c).

In the present invention, the electrode structure refers to the structure of the electrode part 10 configured to be inserted into the human body. When it is combined with a separate main body, it may include the connection part 20 for combining the electrode part 10 with the main body. When the main body is connected to the electrode part 10 in an integrated manner, only the electrode part 10 may be included as the electrode structure.

FIG. 4 shows a partial sectional view and plan view of the electrode structure of a neural probe according to an embodiment of the present invention.

The electrode part 10 of the electrode structure includes a base film 11; electrodes 30 formed on one surface of the base film 11; and an insulation layer 15 partially covering the base film 11 and the electrodes 30.

The base film 11 may be made of a polymer such as polyimide, and may have a thin thickness of 1 mm or less. However, the material or thickness thereof may be changed according to the required conditions. The electrodes 30 and the insulation layer 15 may be formed by applying a semiconductor process to the base film 11.

The electrodes 30 are formed on the base film 11. Although not shown, the wiring 40 connected to the electrodes 30 is also formed. The electrodes 30 are formed in such a manner that a conductive material is deposited on the base film 11, but the manufacturing method is not limited thereto, and various methods may be applied.

The electrodes 30 may have lower impedance when formed to be larger. However, as the size of the electrodes increases, it becomes more difficult to specify a bio-signal measurement target nerve or cell, making it difficult to identify the problem of a specific nerve or cell. In the present embodiment, the electrodes 30 are covered with the insulation layer 15, through holes 16 are formed in the insulation layer 15 to expose parts of the electrodes 30 to the outside, and the electrodes 30 measure or stimulate a nerve or cell through the surfaces exposed as described above, i.e., the exposed surfaces.

The area of the electrodes 30 is larger than the area of the through holes 16, and accordingly, parts of the surfaces of the electrodes 30 are covered with the through holes 16. In the case where the electrodes 30 are circular, the diameter D of the electrodes 30 is larger than the diameter d of the through holes 16. The planar shape of the electrodes 30 is not limited to a circular shape. It is obvious that the electrodes 30 may have various shapes such as a square, a polygon, and an ellipse. It may be preferable that the area of the through holes 16 is 90% or less of the area of the electrodes 30, but the size may be changed depending on the shape of the electrode part 10.

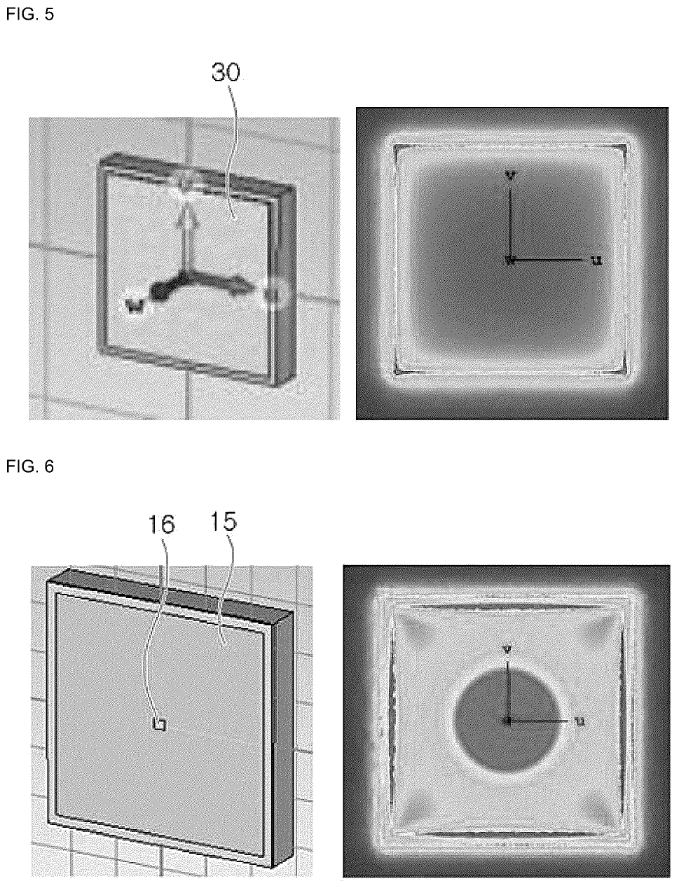

FIGS. 5 and 6 show the results of simulating the current density according to the through holes 16. In the case of FIG. 5, an electrode 30 is not covered with an insulation layer 15, and in FIG. 6, an electrode 30 is covered with an insulation layer 15 and then a through hole 16 is formed. The area of the through hole 16 is approximately 4% of the area of the electrode 30 of FIG. 5. When the experiment was performed under the same conditions, it was found that the current density was high around the through hole 16, as shown in FIG. 6. Compared to that of FIG. 5, a current density approximately 80 times higher than the current density of FIG. 5 was found in the through hole 16 of FIG. 6. Accordingly, accurate stimulation or measurement may be performed at an accurate location.

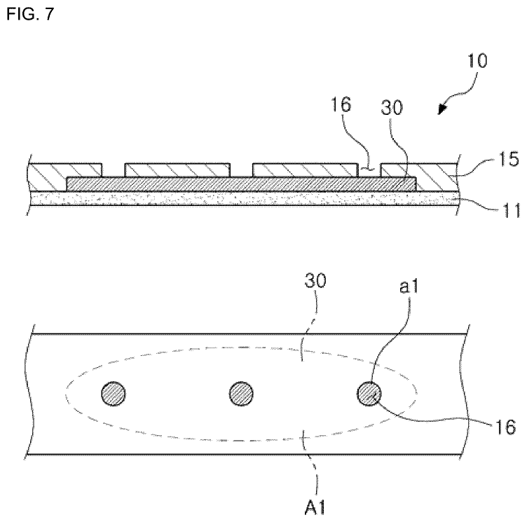

FIG. 7 shows a partial sectional view and plan view of the electrode structure of a neural probe according to another embodiment of the present invention.

The electrode part 10 of the electrode structure includes a base film 11; an electrode 30 formed on one surface of the base film 11; and an insulation layer 15 partially covering the base film 11 and the electrode 30.

Basically, the present embodiment is the same as the embodiment of FIG. 4 except for the electrode 30 and the through holes 16, so that the following description will be given with a focus on the differences therebetween.

The electrode 30 formed on the base film 11 is covered with the insulation layer 15, and the insulation layer 15 includes a plurality of through holes 16 at locations corresponding to the electrode 30. Accordingly, the area A1 of the electrode 30 is larger than the combined area al of the corresponding plurality of through holes 16.

This structure may also increase the current density compared to the case where the overall area of the electrode 30 is exposed without the through holes 16. Accordingly, accurate stimulation or measurement may be performed at an accurate location.

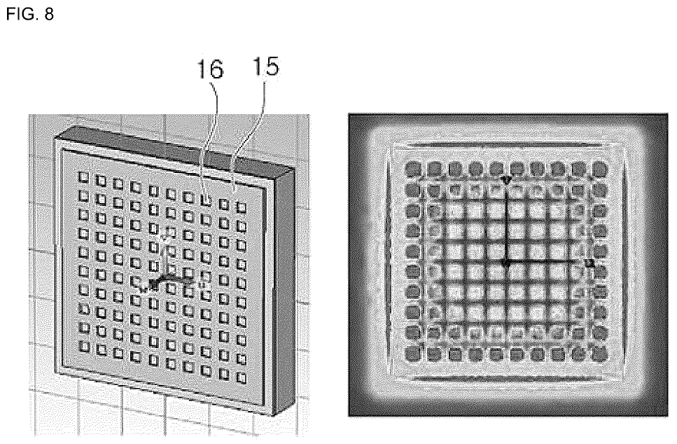

FIG. 8 shows the results of simulating the current density when a plurality of through holes 16 are formed in one electrode. The simulation was performed under the same conditions as the simulation of FIGS. 5 and 6 above. In FIG. 8, the plurality of through holes 16 are formed in the insulation layer 15, and the area of the through holes 16 is approximately 20% of the area of the electrode 30. In this case, the average current density was increased about twice compared to that in the case where the electrode 30 was not covered with the insulation layer 15. Accordingly, even when the plurality of through holes 16 are formed, accurate measurement and stimulation may be performed, and stimulation and bio-signal measurement functions may be implemented in a single neural probe by improving the electrode structure of the neural probe.

As described above, in order to lower the impedance of the electrode 30 in an embodiment of the present invention, the size of the electrode 30 may be configured to be as large as possible, and only an exposed area may be configured to be small. Since the impedance of the electrode decreases as the contact area of the electrode increases, it is assumed that the impedance is lowered by increasing the size of the electrode, and thus, an environment in which high energy can be transmitted is constructed. In particular, when only an exposed area is configured to be small, high energy may be concentrated, generated and transmitted from an electrode having low impedance.

The sectional view of the electrode probe coated with the electrical insulation layer so that the size of the exposed area is smaller than the size of the electrode(s) is shown for each of the embodiments of FIG. 4 and FIG. 7. When only part of the electrode is exposed in this manner, the stimulation energy of the electrode for bio-stimulation is particularly concentrated on the exposed area, so that energy having a high stimulation value is intensively transmitted to a local nerve or cell, thereby performing effective stimulation. Furthermore, a plurality of nerves or cells may be stimulated with high energy at the same time by providing a plurality of exposed areas.

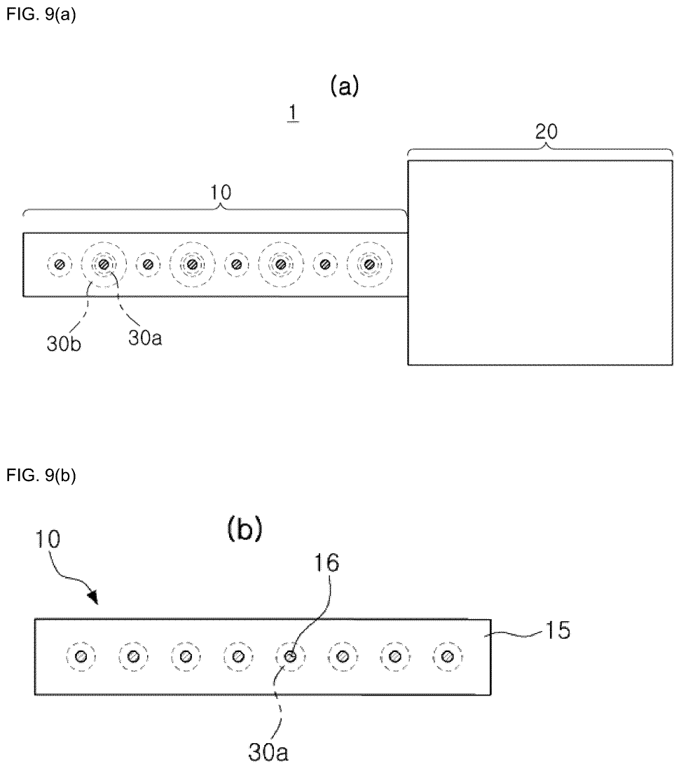

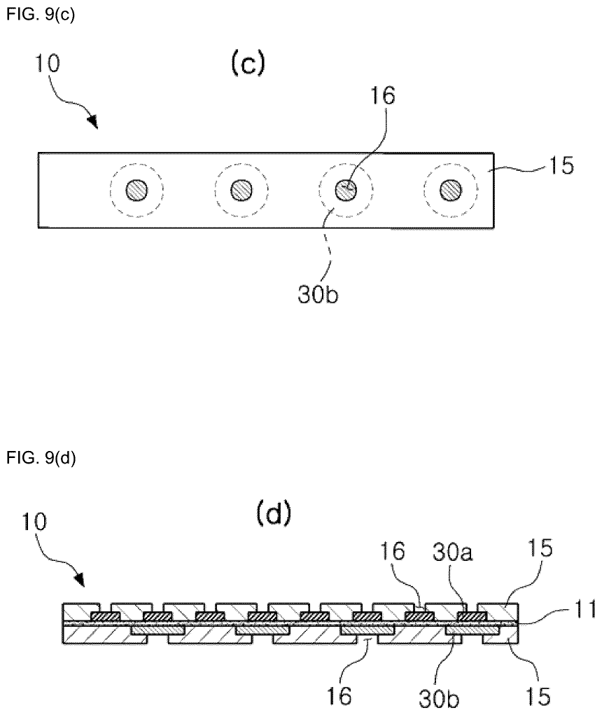

Meanwhile, FIG. 9 shows a schematic diagram of an embodiment of an electrode part 10 including measurement electrodes 30a and stimulation electrodes 30b.

As shown in FIG. 9, the electrode structure is formed in an electrode part 10. In the electrode part 10, the measurement electrodes 30a connected to a measurement circuit 70 (see FIG. 13) are disposed on one surface of a base film 11, and the stimulation electrodes 30b connected to a stimulation circuit 80 (see FIG. 13) are disposed on the other surface of the base film 11. The measurement electrodes 30a and the stimulation electrodes 30b are partially covered with an insulation layer 15 and partially exposed via through holes 16. The base film 11 between the measurement electrodes 30a and the stimulation electrodes 30b are covered with the insulation layer 15, so that electrical conduction does not occur between each electrode and its neighboring electrode.

Although the electrodes are arranged in a linear-type arrangement in the present embodiment, the arrangement of the electrodes is not limited thereto. It is obvious that the electrodes are arranged in a tetrode-type arrangement.

Furthermore, at least one measurement electrode 30a and at least one stimulation electrode 30b may be arranged on different surfaces, but may be arranged at corresponding locations. In this case, there may be provided an electrode structure for a neural probe that allows a bio-signal measurement location and a stimulation location to match each other as much as possible. Using the electrode structure of one neural probe, a bio-signal may be measured from a surrounding nerve or cell and a bio-stimulation may be applied to the corresponding nerve or cell, simultaneously or sequentially.

Furthermore, compared to the conventional case of separately operating a neural probe for measuring a bio-signal and a neural probe for applying bio-stimulation, an invasive range is reduced. Furthermore, there may be easily overcome a problem in which an error (for example, an error in which bio-stimulation is applied to a nerve or cell different from the nerve or cell from which a bio-signal was measured) occurs when measuring a bio-signal from a nerve or cell and stimulating the corresponding nerve or cell.

In this case, it is preferable that at least one pair of the measurement electrode 30a and the stimulation electrode 30b are formed at overlapping locations on both surfaces. This is intended to prevent an error in which bio-stimulation is applied to a nerve or cell different from a bio-signal measurement target nerve or cell by maintaining correspondence between the bio-signal measurement target nerve or cell and a bio-stimulation target nerve or cell as much as possible. In particular, the at least one pair of electrodes formed at the overlapping locations have a concentric structure, and have the advantage of being able to exactly match a bio-signal measurement target nerve or cell and a bio-stimulation target nerve and or cell.

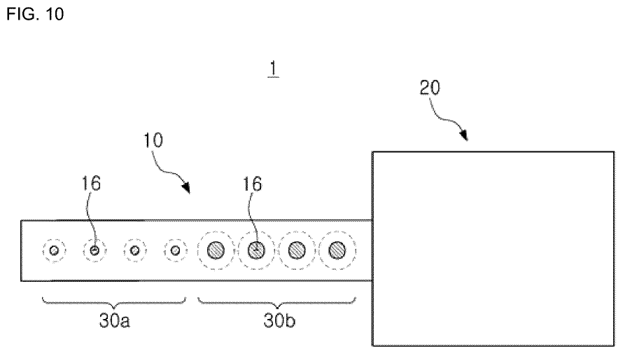

FIG. 10 shows a schematic diagram of another embodiment of an electrode part 10 including measurement electrodes 30a and stimulation electrodes 30b.

As shown in FIG. 10, an electrode structure is formed in the electrode part 10. In the electrode part 10, the measurement electrodes 30a connected to the measurement circuit 70 (see FIG. 13) and the stimulation electrodes 30b connected to the stimulation circuit 80 (see FIG. 13) are arranged on one surface of a base film 11. Although not shown, the measurement electrodes 30a and the stimulation electrodes 30b are partially covered with an insulation layer 15 and partially exposed by through holes 16. The base film 11 between the measurement electrodes 30a and the stimulation electrodes 30b is covered with the insulation layer 15, so that electrical conduction does not occur between each electrode and its neighboring electrode.

When formed on the same surface, the two types of electrodes may be arranged according to a predetermined rule, or may be arranged randomly. Since the individual electrodes may be driven through individual operation control regardless of the type of arrangement, any arrangement is fine. However, it is preferable that each pair of a stimulation electrode 30b and a bio-signal measurement electrode 30a are configured to be close to each other.

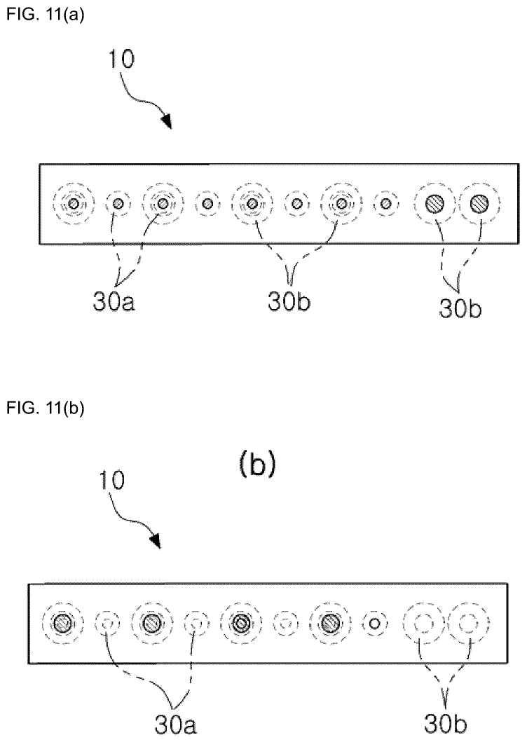

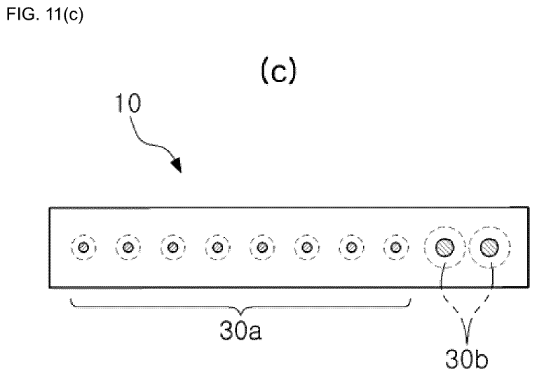

FIG. 11 shows a schematic diagram of another embodiment of an electrode part 10 including measurement electrodes 30a and stimulation electrodes 30b.

As shown in FIG. 11, an electrode structure is formed in the electrode part 10. In the electrode part 10, measurement electrodes 30a connected to the measurement circuit 70 (see FIG. 13) and some of stimulation electrodes 30b connected to the stimulation circuit 80 (see FIG. 13) are arranged on one surface of a base film 11, and the remainder of the stimulation electrodes 30b are arranged on the other surface of the base film 11. The base film 11 between the measurement electrodes 30a and the stimulation electrodes 30b is covered with an insulation layer 15, so that electrical conduction does not occur between each electrode and its neighboring electrode.

As in FIG. 9, each measurement electrode 30a and each stimulation electrode 30b may be arranged at corresponding locations when they are located on different surfaces.

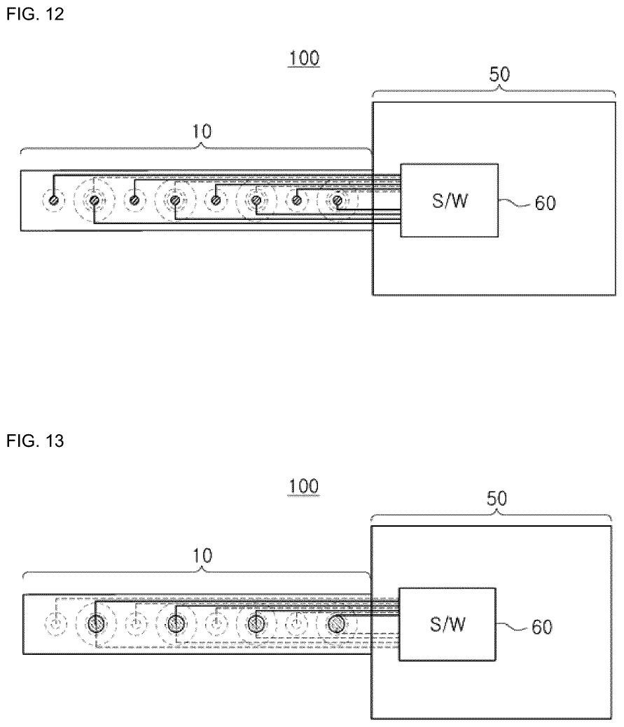

FIGS. 12 and 13 show schematic diagrams of the electrode module of a neural probe. However, it is not limited to the main body 50 of the electrode module. An electrode part 10 may be connected to a connection part, and may be configured in the same manner as in the case of the main body even when connected to the connection part 20. In the present embodiment, for example, a switching means or switching element 60 may be provided in the main body 50, and may be connected to at least bio-signal measurement electrodes or bio-stimulation electrodes.

For example, bio-signal measurement and bio-stimulation may be selectively performed by connecting the switching element 60 to the measurement electrodes 30a and the stimulation electrodes 30b and performing selective switching. That is, each of the electrodes 30a and 30b may be connected to the switching element 60, and may be individually controlled to be turned on and off.

Furthermore, for example, the measurement electrodes 30a may be individually connected to the switching element 60, and the stimulation electrodes 30b may be connected in parallel with each other and then the final electrode may be connected to the switching element 60. The switching element 60 may be connected to the bio-stimulation electrode closest to the switching element 60 among the bio-stimulation electrodes 30b, and the other stimulation electrodes 30b may be connected in parallel to the switching means 60. As described above, the stimulation electrodes are connected in parallel with each other, so that there is an advantage in that the impedance acting on the electrodes may be lowered, and thus, a nerve or cell may be stimulated more effectively over a wider stimulation range.

Meanwhile, the measurement electrodes 30a may be individually connected to the switching element 60.

As a result of configuring both the bio-signal measurement electrodes and the bio-stimulation electrodes in a single neural probe, the neural probe may be operated as follows.

The operation may include the step of measuring the bio-signal of a nerve or cell by supplying power to the measurement electrodes 30a via the switching element 60; the step of comparing the value measured from the measured bio-signal with a reference value used to determine whether to perform stimulation; and the step of applying stimulation to the nerve or cell by supplying power to the stimulation electrodes 30b via the switching element 60 when the value measured from the bio-signal is equal to or higher than the reference value.

That is, the reference value may be, for example, a reference value that determines the necessity of bio-stimulation. This depends on the results of bio-signal measurement for a specific nerve or cell. When a bio-signal value that can be obtained as a result of bio-signal measurement is lower than or equal to a minimum reference value or equal to or higher than a maximum reference value, a corresponding nerve or cell is determined to be abnormal, and predetermined stimulation may be applied to the nerve or cell. The intensity or duration of the stimulation may be adjusted, and may be determined according to the results of the bio-signal measurement.

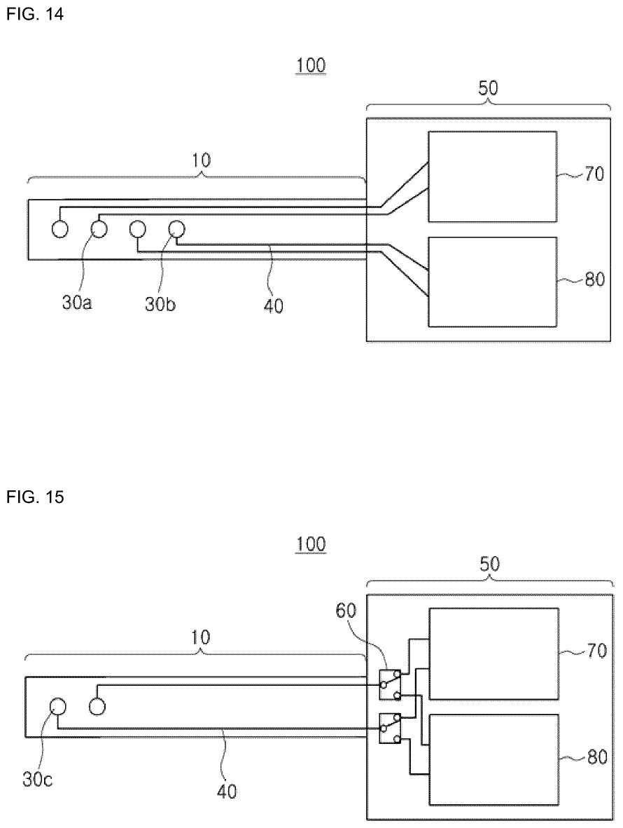

FIGS. 14 to 18 disclose embodiments of electrode modules.

An electrode module 100 corresponds to a concept that includes an electrode part 10, and a main body 50 including a circuit part. A bio-signal measurement and stimulation device may be configured by adding peripheral components such as a power supply part for supplying power to an electrode module, a communication part, and/or the like.

As shown in FIG. 14, the electrode module 100 includes the electrode part 10, and the main body 50 connected to the electrode part 10 and including the circuit part. The main body 50 may be configured by forming the circuit part on the same substrate, i.e., the base film 11 (see FIG. 4), as the electrode part 10. The electrode part 10 and the main body 50 may be configured on a single base film. The circuit part includes a measurement circuit 70 and a stimulation circuit 80. In FIG. 14, measurement electrodes 30a and stimulation electrodes 30b are formed together on one surface of the electrode part 10. The measurement electrodes 30a are connected to the measurement circuit 70 through a wiring 40, and the stimulation electrodes 30b are connected to the stimulation circuit 80 through a wiring 40.

In the present embodiment, the electrodes 30a and 30b and the circuits 70 and 80 are formed on the one base film, so that the size can be reduced, and the measurement electrodes 30a and the stimulation electrodes 30b are arranged on the one electrode part 10, so that measurement and stimulation can be performed through the one electrode part 10.

In the case of FIG. 15, there is shown an electrode module 100 in which an electrode part 10 and a main body 50 are formed on a single base film, as in the case of FIG. 14.

In FIG. 15, the electrodes 30c of the electrode part 10 are connected to a measurement circuit 70 and a stimulation circuit 80 through a wiring 40 and switching elements 60. The switching elements 60 are separate elements located between the electrodes 30c, and the measurement circuit 70 and the stimulation circuit 80, and connect the electrodes 30c to one of the measurement circuit 70 and the stimulation circuit 80 depending on the situation or in response to a signal. That is, the electrodes 30c of the electrode part 10 are measurement and stimulation electrodes 30c that alternately perform measurement and stimulation.

The switching elements 60 switch the circuits connected to the measurement and stimulation electrodes 30c according to a preset algorithm or a signal from a communication unit connected to the electrode module 100. The individual electrodes 30c are connected to different switching elements 60, respectively, so that one measurement and stimulation electrode 30c can perform measurement and its neighboring measurement and stimulation electrode 30c can perform stimulation. Alternatively, they may also perform measurement and stimulation together.

In the case of FIG. 15, measurement and stimulation are performed with the same electrodes 30c, so that it is possible to provide stimulation at the same location where the measurement was performed. Accordingly, there may be prevented an error in which bio-stimulation is applied to a nerve or cell different from a bio-signal measurement target nerve or cell by maintaining correspondence between a bio-signal measurement target nerve or cell and a bio-stimulation target nerve and or cell. In particular, measurement and stimulation are performed with the same electrodes, so that there is an advantage of being able to exactly match a signal measurement target nerve or cell and a stimulation target nerve or cell.

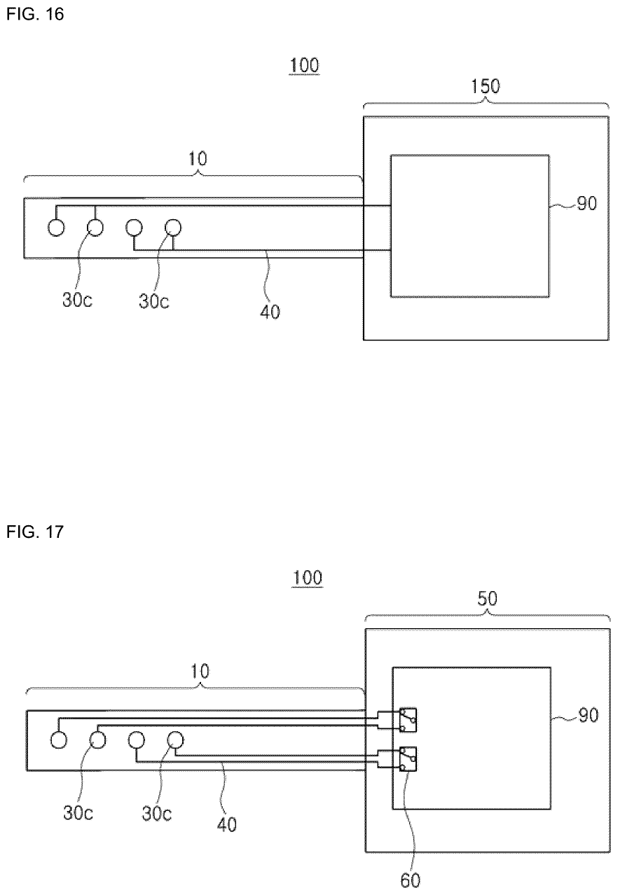

FIGS. 16 and 17 are modifications of the embodiment of FIG. 15. In the case of FIG. 16, measurement and stimulation electrodes 30c are connected to the measurement and stimulation circuit 90 of a main body 50. In this case, although there is no switching element 60, the measurement and stimulation electrodes 30c are connected to the measurement and stimulation circuit 90 in which the circuit can be converted into a measurement circuit and a stimulation circuit, so that measurement and stimulation can be performed with the same electrodes 30c as in FIG. 15. The measurement and stimulation circuit 90 refers to a circuit capable of performing at least the roles of a measurement circuit and a stimulation circuit, and may also perform roles other than the roles of a measurement circuit and a stimulation circuit. Furthermore, the electrodes 30c are connected in parallel to the circuit. In this case, there is an advantage in that the impedance acting on the electrodes 30c can be lowered by connecting the electrodes 30c in the state of being parallel with each other. Based on this, a nerve or cell may be stimulated more effectively over a wider stimulation range.

In the case of FIG. 17, there is shown an electrode module 100 in which stimulation electrodes 30c are connected to the measurement and stimulation circuit 90 of a main body 50, as in the case of FIG. 16. In the embodiment of FIG. 17, switching elements 60 are located in the measurement and stimulation circuit 90, and the switching elements 60 determine the electrodes 30c to be connected to the circuit. In the case where the measurement and stimulation circuit 90 performs the role of a measurement circuit, the electrodes 30c to perform measurement may be controlled through the switching elements 60.

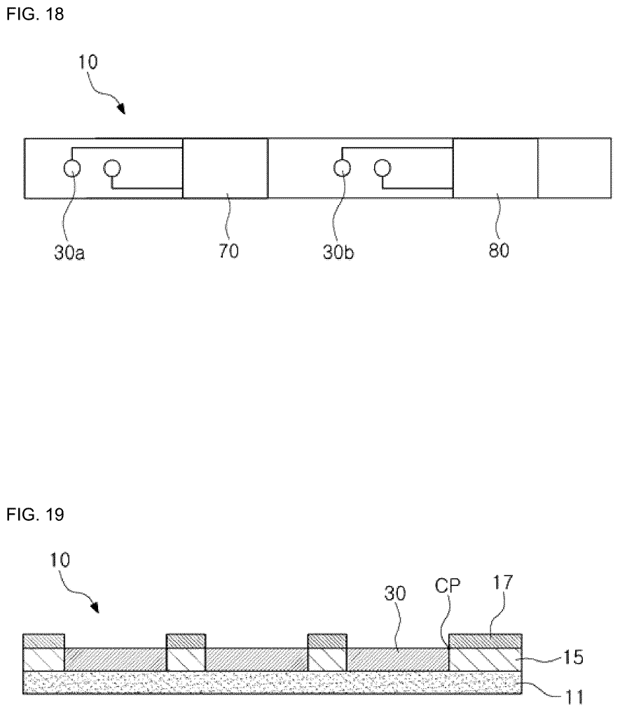

Meanwhile, FIG. 18 shows a structure in which a measurement circuit 70 and a stimulation circuit 80 are arranged in an electrode part 10 without the main body 50.

As shown in FIG. 18, the electrode part 10 includes measurement electrodes 30a and stimulation electrodes 30b formed on a base substrate. On the same base substrate, a measurement circuit 70 is formed between the measurement electrodes 30a and the stimulation electrodes 30b, and a stimulation circuit 80 is formed near the stimulation electrodes 30b. The measurement electrodes 30a are connected to the measurement circuit 70 through a wiring 40, and the stimulation electrodes 30b are connected to the stimulation circuit 80 through a wiring 40.

As described above, in the present invention, the circuits and the electrodes are formed on the base substrate. Accordingly, the sizes of the circuits are made sufficiently small and the circuits are formed in the electrode part 10 to be inserted into a nerve or a cell, so that a separate main body for the stimulation circuit and the measurement circuit is unnecessary. This may reduce the burden on the human body by allowing the overall device to be manufactured in a small size.

Although not shown here, it is obvious that the measurement and stimulation circuit 90 may be formed in the electrode part 10 and the switching elements 60 may also be formed in the electrode part 10, as shown in FIGS. 15 and 16.

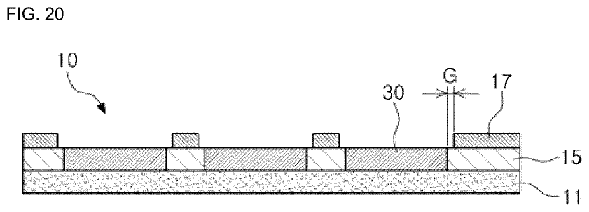

FIGS. 19 and 20 show sectional views of electrode structures.

In the case of electrodes 30, they operate when power is applied. During an operation process, heat is generated. In order to effectively dissipate such heat, in an electrode structure, an electrode part 10 further includes a heat dissipation layer 17 in the present embodiment. The heat dissipation layer 17 may be configured to come into contact with electrodes 30 having relatively high temperature and directly dissipate heat from the electrodes 30, or may be configured to move and dissipate heat from areas near the electrodes 30 even without coming into direct contact with the electrodes 30.

FIG. 19 shows a sectional view of the electrode part 10 including the heat dissipation layer 17 that is an electrical insulator but has high thermal conductivity. As shown in FIG. 19, the electrodes 30 and an insulation layer 15 are formed on a base film 11, and the heat dissipation layer 17 is formed on the insulation layer 15. A thermally conductive ceramic that is both highly thermally conductive and electrically insulating may be applied as the heat dissipation layer 17, but the heat dissipation layer 17 is not limited thereto. In the case of the electrically insulating heat dissipation layer 17, a short circuit does not occur even when it comes into contact with the electrode 30, so that a contact portion CP may be provided, as shown in FIG. 19. Furthermore, the heat dissipation layer 17 may cover parts of the electrodes 30 like the insulation layer 15 of FIG. 7, and it may also be possible to cover parts of the base film 11 and the electrodes 30 with the heat dissipation layer 17 instead of the insulation layer 15.

That is, the heat dissipation layer according to an embodiment of the present invention may be formed to replace part of an electrical insulation layer formed adjacent thereto. The heat dissipation layer may come into contact with the side surfaces of electrodes, receive heat from the electrodes, and dissipate it to the outside, and may be made of an electrically non-conductive material. The heat dissipation layer is electrically non-conductive. Accordingly, although not shown, the heat dissipation layer may replace the overall electrical insulation layer.

FIG. 20 shows a sectional view of the electrode part 10 including the heat dissipation layer 17 having high thermal conductivity and high electrical conductivity. The electrode part 10 is the same as that of the embodiment of FIG. 19 in that electrodes 30 and an insulation layer 15 are formed on a base film 11 and the heat dissipation layer 17 is formed on the insulation layer 15. However, the heat dissipation layer 17 of FIG. 19 has electrical conductivity, and thus, a short circuit may occur when it comes into contact with the electrode 30. Accordingly, the heat dissipation layer 17 is formed on the insulation layer 15 to be spaced apart from the electrodes 30 by a predetermined distance G. This heat dissipation layer 17 may be a metal layer.

This heat dissipation layer 17 absorbs the heat, generated from the electrodes 30, from areas near the electrodes 30, and dissipates it to the outside.

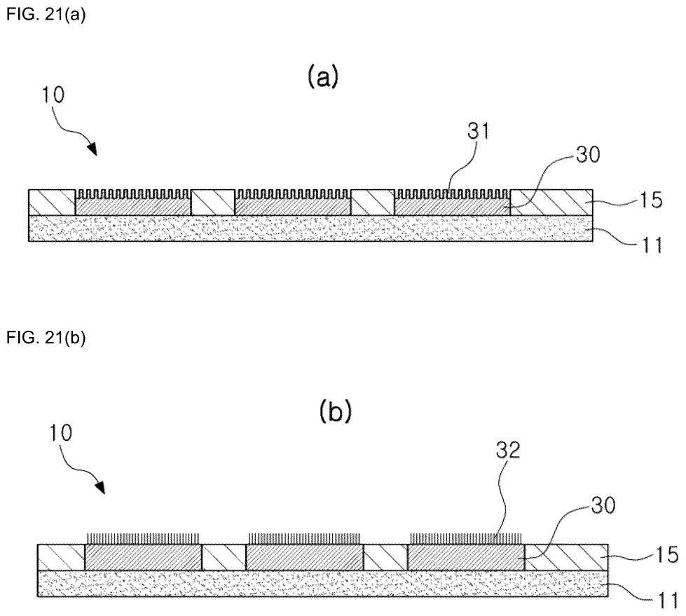

Furthermore, the present invention may be surface-processed to increase the surface areas of electrodes. FIG. 21 shows a sectional view of an electrode part 10 in which depression-protrusion structures are formed to increase surface areas. There is an effect in that when the surface areas of electrodes 30 increase, the impedance of the electrodes 30 is lowered.

The purpose of surface processing is to increase the surface areas of the electrodes. This is based on the same principle as increasing the sizes of the electrodes 30. The surface processing method described above is not limited to a particular method. As shown in FIG. 21(a), the electrodes 30 and an insulation layer 15 may be formed at the same height, and then a depression portion 31 may be formed on the surface of each of the electrodes 30. In the present embodiment, the depression portions 31 have a lower height than the insulation layer 15, so that depression-protrusion structures are formed on the surfaces of the electrodes 30. The depression portions 31 may be formed by a surface etching or surface roughening method or the like.

In FIG. 21(b), the electrodes 30 and an insulation layer 15 are formed at the same height, and then a protrusion portion 32 may be formed on the surface of each of the electrodes 30. In the present embodiment, the protrusion portions 32 have a higher height than the insulation layer 15, so that depression-protrusion structures are formed. The protrusion portions 32 may be formed by a nanowire growth, deposition, or plating method, or the like. The structures formed by such a method are also called the depression-protrusion structures in the present invention.

Meanwhile, forming depression-protrusion structures on the electrodes 30 is not limited to the case where the insulation layer 15 and the electrodes 30 are formed at the same height, and may be applied equally to the case where the insulation layer 15 covers parts of the electrodes 30, as shown in FIG. 4. In this case, it would be sufficient if the depression-protrusion structures are formed only on the exposed surfaces of the electrodes 30. However, it is also acceptable if the insulation layer 15 covering parts of the electrodes 30 is formed after depression-protrusion structures have been formed on the overall outer surfaces of the electrodes 30.

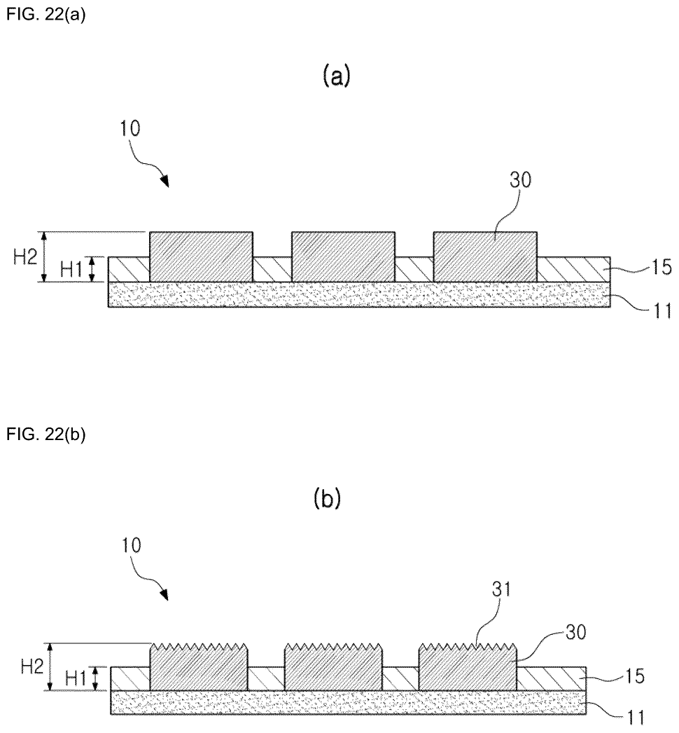

FIG. 22 shows a sectional view of the electrode structure of a neural probe according to another embodiment of the present invention.

As shown in FIG. 22, electrodes 30 and an insulation layer 15 are formed on a base film 11. The electrodes 30 are formed to have a higher height than the insulation layer 15. That is, the height H2 of the electrodes 30 is higher than the height H1 of the insulation layer 15 on the base film 11.

The height H2 of the electrodes 30 is higher than the height H1 of the insulation layer 15. Each of the electrodes 30 may easily come into contact with a nerve or cell, and the contact area between the electrode and the nerve or cell may be increased, which also provides the effect of lowering the impedance.

In FIG. 22(a), there is shown an embodiment in which electrodes 30 are higher than an insulation layer 15. In FIG. 22(b), there is shown an embodiment in which electrodes 30 are higher than an insulation layer 15 and one surface of each of the electrodes 30 is surface-processed to form a depression-protrusion structure.

As shown in FIG. 22(b), when the electrodes 30 are formed to be higher than the insulation layer 15 and one surface of each of the electrodes 30 is surface-processed to form a depression-protrusion structure, the contact area between the electrode 30 and a nerve or cell may be further increased.

Although the present invention has been described in more detail above with reference to the embodiments, the present invention is not necessarily limited to these embodiments, but various modifications may be made within a scope that does not depart from the technical spirit of the present invention. Therefore, the embodiments disclosed in the present invention are not intended to limit the technical spirit of the present invention but are intended to describe it, and the scope of the technical spirit of the present invention is not limited by these embodiments. The scope of protection of the present invention should be interpreted based on the attached claims, and all technical spirits within the equivalent scope should be interpreted as being included in the scope of the rights of the present invention.

1. An electrode structure for a neural probe that measures bio-logical signals or applies stimulation, the electrode structure comprising:
a substrate;
electrodes formed on at least one surface of the substrate; and
a wiring formed on the substrate and connected to the electrodes;
wherein the electrodes comprise one or more measurement electrodes connected to a measurement circuit and one or more stimulation electrodes connected to a stimulation circuit.

2. The electrode structure of claim 1, wherein the measurement electrodes are arranged on one surface of the substrate, and the stimulation electrodes are arranged on an opposite surface of the base film.

3. The electrode structure of claim 2, wherein at least some of the measurement electrodes and at least some of the stimulation electrodes are arranged at corresponding locations with the substrate interposed therebetween.

4. An electrode structure for a neural probe that measures bio-logical signals or applies stimulation, the electrode structure comprising:
a substrate;
electrodes formed on at least one surface of the substrate; and
a wiring formed on the substrate and connected to the electrodes;
wherein the electrodes comprise one or more measurement and stimulation electrodes connected to both a measurement circuit and a stimulation circuit.

5. The electrode structure of claim 4, wherein switching elements configured to selectively allow and block connection between the measurement and stimulation electrodes and the measurement circuit or the stimulation circuit are arranged between the measurement circuit and the stimulation circuit, and the measurement and stimulation electrodes.

6. The electrode structure of claim 1, further comprising a heat dissipation layer exposed out of an outer surface of the electrode structure.

7. The electrode structure of claim 6, wherein a material of the heat dissipation layer is electrically conductive, and the heat dissipation layer is arranged apart from the electrodes.

8. The electrode structure of claim 6, wherein a material of the heat dissipation layer is electrically non-conductive.

9. The electrode structure of claim 8, wherein parts of the heat dissipation layer come into contact with the electrodes.

10. The electrode structure of claim 1, further comprising an insulation layer configured to cover parts of the substrate and the electrodes.

11. The electrode structure of claim 10, wherein the electrodes are higher than the insulation layer.

12. The electrode structure of claim 11, wherein surfaces of the electrodes each have a depression-protrusion structure.

13. The electrode structure of claim 10, wherein the insulation layer comprises through holes exposing the electrodes, and an area of the through holes is smaller than an area of the corresponding electrodes.

14. The electrode structure of claim 13, wherein at least some of the electrodes correspond to the plurality of through holes, and a sum of areas of the corresponding through holes is smaller than an area of the electrodes.

15. The electrode structure of claim 10, further comprising a heat dissipation layer disposed on the insulation layer and exposed out of an outer surface of the electrode structure.

16. The electrode structure of claim 1, wherein surfaces of the electrodes each have a depression-protrusion structure.

17. The electrode structure of claim 16, wherein:
the electrodes further comprise an insulation layer covering spaces between the substrate and the electrodes; and
the surfaces of the electrodes each comprise a depression portion configured to be lower than the insulation layer.

18. The electrode structure of claim 16, wherein:
the electrodes further comprise an insulation layer covering spaces between the substrate and the electrodes; and
the surfaces of the electrodes each comprise a protrusion portion configured to be higher than the insulation layer.

19. The electrode structure of claim 1, further comprising:
the measurement circuit formed on the substrate and connected to the measurement electrodes; and
the stimulation circuit formed on the substrate and connected to the stimulation electrodes.

20. The electrode structure of claim 1, wherein at least parts of the wiring connect the plurality of electrodes in parallel with each other.

21. An electrode module for a neural probe, the electrode module comprising:
the electrode structure for a neural probe set forth in claim 1; and
a main body comprising a circuit part connected to the electrodes of the electrode structure;
wherein the circuit part and the electrode structure are formed on the same substrate.

22. The electrode module of claim 21, wherein:
the measurement electrodes are arranged on one surface of the substrate, and the stimulation electrodes are arranged on an opposite surface of the base film; and
the measurement circuit is arranged on the one surface in the main body, and the stimulation circuit is arranged on the opposite surface in the main body.

23. The electrode module of claim 21, wherein:
at least some of the measurement electrodes and the stimulation electrodes are arranged on one surface of the substrate; and
the measurement circuit and the stimulation circuit are arranged on the one surface in the main body.

24. An electrode module for a neural probe, the electrode module comprising:
the electrode structure for a neural probe set forth in claim 4; and
a main body comprising a circuit part connected to the measurement and stimulation electrodes;
wherein the circuit part comprises the measurement circuit and the stimulation circuit.

25. The electrode module of claim 24, wherein the main body further comprises switching elements connecting the circuit part and the electrode structure.

26. The electrode module of claim 25, wherein the switching elements are arranged together with the circuit part.

27. The electrode module of claim 25, wherein the switching elements are arranged between the circuit part and the electrodes.
